(19) United States
(12) Patent Application Publication
(10) Pub. No.: US 2013/0143829 A1
(43) Pub. Date: Jun. 6, 2013

(54) Topical Combination Formulations of Macrocyclic Lactones with Synthetic Pyrethroids

(71) Applicant: Robert Holmes, Burkenhead (NZ)

(72) Inventor: Robert Holmes, Burkenhead (NZ)

(21) Appl. No.: 13/312,321

(22) Filed: Dec. 6, 2011

(51) Int. Cl. A61K 36/287; A61P 33/00; A61K 31/7048

(52) U.S. Cl. 514/30; 514/66

(57) ABSTRACT

The present invention provides pyrethroid/macrocyclic lactone-containing topical formulations that are effective against animal pests, including ecto- and endoparasites. The formulations may be used for combating parasites in or on birds and mammals. The invention also provides for an improved method for eradicating, controlling and preventing parasite and/or pest infestation of birds and mammals.

### FIELD OF THE INVENTION

The present invention is directed to new topical parasiticidal formulations comprising pyrethroids and macrocyclic lactones. The present invention also provides methods for eradicating, controlling, and preventing parasite infestation and/or infection in or on birds, fish and mammals. The formulations of the invention may be administered to animals, particularly mammals, fish and birds, to prevent or treat parasitic infestation and/or infection.

### BACKGROUND OF THE INVENTION

Animals, such as mammals, fish and birds are often susceptible to parasite infestation and/or infection. These parasites may be ectoparasites, such as insects and acarine species, and endoparasites such as filariae and other worms. Endoparasites pose a significant problem to those who raise production animals such as bovines, ovines, and porcines. Thus, there is an ongoing need to develop active pesticidal and parasiticidal compounds to protect animals against attack or infestation/infection by pests.

Many endoparasiticides or anthelmintics exist in the art for treating internal parasites and other animal pests. These endoparasiticides vary in cost as well as their effectiveness to a particular parasite. However, the results of treatment with these pesticides are not always satisfactory because of continued development of resistance by the parasite to the available therapeutic agents. Thus, there is a need in the art for more effective parasiticidal agents for treatment and protection of animals, e.g. mammals, fish and birds from infestation by animal parasites.

The avermectin and milbemycin series of compounds are potent anthelmintic and antiparasitic agents against a wide range of internal and external parasites. The compounds which belong to this series are either natural products or are semi-synthetic derivatives thereof. The structure of these two series of compounds are closely related and they both share a complex 16-membered macrocyclic lactone ring; however, the milbemycins do not contain the disaccharide substituent in the 13-position of the lactone ring. The natural product avermectins are disclosed in U.S. Pat. No. 4,310,519 to Albers-Schonberg, et al., and the 22, 23-dihydro avermectin compounds are disclosed in Chabala, et al., U.S. Pat. No. 4,199,569. For a general discussion of avermectins, which include a discussion of their uses in humans and animals, see “Ivermectin and Abamectin,” W. C. Campbell, ed., Springer-Verlag, N.Y. (1989). Naturally occurring milbemycins are described in Aoki et al., U.S. Pat. No. 3,950,360 as well as in the various references cited in “The Merck Index” 12^{th} ed., S. Budavari, Ed., Merck & Co., Inc. Whitehouse Station, N.J. (1996). Semi-synthetic derivatives of these classes of compounds are well known in the art and are described, for example, in U.S. Pat. No. 5,077,308, U.S. Pat. No. 4,859,657, U.S. Pat. No. 4,963,582, U.S. Pat. No. 4,855,317, U.S. Pat. No. 4,871,719, U.S. Pat. No. 4,874,749, U.S. Pat. No. 4,427,663, U.S. Pat. No. 4,310,519, U.S. Pat. No. 4,199,569, U.S. Pat. No. 5,055,596, U.S. Pat. No. 4,973,711, U.S. Pat. No. 4,978,677, and U.S. Pat. No. 4,920,148, all incorporated herein by reference in their entirety. A particularly useful macrocyclic lactone is eprinomectin, which is described in U.S. Pat. No. 6,174,540, U.S. Pat. No. 6,733,767, U.S. Pat. No. 5,602,107 to Merck (all incorporated by reference). This compound provides for a very short milk withdrawal period due to it tendency to partition away from the milk of a lactating mammal.

To overcome the problem of resistance, various combinations of anthelmintics have been extensively researched. Unfortunately, combinations of macrocyclic lactone anthelmintics with anthelmintics of other families have proven difficult to formulate. For example, Closamectin (injectable, manufactured by Norbrook) contains ivermectin at 0.5% and closantel at 12.5% and is so viscous that the product is supplied in a special pack that has a warming device. The warming device is activated 15 minutes before administration and the pack requires periodic shaking throughout this time to ensure temperature reduces the viscosity sufficiently for acceptable administration. Another example is combining a macrocyclic lactone (ML) with levamisole. The difficulties of combining these two actives lies in the stability of each being incompatible in the environment in which the other is stable. Levamisole is stable in acid conditions usually a pH of about 4 or below, while macrocyclic lactones are unstable in conditions where the pH is acidic. Both levamisole and macrocyclic lactones are well known in the art. In particular, levamisole has been used as an anthelmintic to treat nematodes and respiratory worm infestations in both humans and animals. Levamisole may be combined with other actives, although often with significant difficulty, as is described in various patents and applications (for example, US 2009/0075918 A1 to Neto et al.; WO 00/061068, which describes non-aqueous pour-on formulations that further contain triclabendazole, and WO 04/009080, which describes stable pyrrolidone-based formulations). Finally, AU200310102 (to Nufarm and Novartis) describes formulations having levamisole, avermectins, and benzimidazole, said formulations appearing to make use of a biphasic solvent system, in an attempt to overcome the problematic instability of formulations which comprise levamisole and macrocyclic lactones.

Another class of parasiticidal actives known in the art are the pyrethroids. A pyrethroid is a synthetic chemical compound similar to the natural chemical pyrethrins produced by the flowers of pyrethrums (C. cinerariaefolium and C. coccineum). Pyrethroids now constitute a major proportion of the synthetic insecticide market and are common in commercial products such as household insecticides. Pyrethroids are usually broken apart by sunlight and the atmosphere in one or two days, and do not significantly affect groundwater quality, and these compounds tend to yield low residues in the milk of dairy cattle. After reviewing the current literature, applicants are unaware of any previously described, effective combinations of pyrethroids and eprinomectin, as topical formulations or otherwise.

It is expressly noted that citation or identification of any document in this application is not an admission that such document is available as prior art to the present invention. Any foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### SUMMARY OF THE INVENTION

The invention provides novel parasiticidal formulations comprising pyrethroids and macrocyclic lactones, particularly avermectins such as ivermectin and eprinomectin, and milbemycins, such as moxidectin, milbemectin, and milbemycin oxime. The formulations are storage-stable and provided in several solvent systems. In one embodiment, the actives are both dissolved in a solvent. In another embodiment, one active is dissolved in a solvent and the other active is suspended in an aqueous phase. The invention also provides methods for the treatment and prevention of parasitic infestation and/or infection of animals.

The inventive formulations comprising the pyrethroids and macrocyclic lactones are highly effective for the treatment or prophylaxis of parasites in or on mammals, fish and birds, and in particular, cats, dogs, horses, chickens, pigs, sheep and cattle with the aim of ridding these hosts of all the parasites commonly encountered by mammals, fish and birds. The invention also provides for effective and long-lasting defense against endoparasites, such as helminths, nematodes, filariae, hookworms, whipworms and roundworms of the digestive tract of animals and humans.

Accordingly, the present invention provides methods for preventing and treating parasites in or on animals, comprising administering a parasiticidally effective amount of a storage-stable topical formulation comprising pyrethroids and macrocyclic lactone to the animal. The invention also provides a method for combating or controlling animal pests and parasites.

It is an object of the invention to not encompass within the invention any previously known product, process of making the product, or method of using the product such that the Applicants reserve the right to this invention and hereby disclose a disclaimer of any previously known product, process, or method.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law; e.g., they allow for elements not explicitly recited, but exclude elements that are found in the prior art or that affect a basic or novel characteristic of the invention.

These and other embodiments are disclosed or are obvious from, and encompassed by, the following Detailed Description.

### BRIEF DESCRIPTION OF DRAWINGS

The following Detailed Description, given by way of example, and not intended to limit the invention to specific embodiments described, may be understood in conjunction with the accompanying Figures, incorporated herein by reference, in which:

FIG. 1 depicts mean eprinomectin (mg/kg) in milk at time points post treatment

FIG. 2 depicts mean deltamethrin (mg/kg) in milk at time points post treatment

FIG. 3 depicts mean plasma eprinomectin (mg/kg) at time points post treatment with EPRINEX, inventive suspension, and inventive solution

### DETAILED DESCRIPTION

This invention relates, in part, to topical combination anthelmintic formulations of macrocyclic lactones and synthetic pyrethroids for application to cattle.

In some embodiments the macrocyclic lactone active is abamectin, doramectin, eprinomectin, ivermectin, moxidectin, milbemycin, milbemectin, latidectin, or the like, or combinations thereof.

In some embodiments, the synthetic pyrethroid active is cyfluthrin, cypermethrin, deltamethrin, flumethrin, or the like, or combinations thereof.

In one embodiment, the formulation is a combination of eprinomectin and deltamethrin containing 5 g/L eprinomectin and 7.5 g/L deltamethrin designed for topical delivery to the backline of cattle at a dose rate of 1 mL per 10 kg animal body weight.

In an embodiment, inventive formulations comprising eprinomectin and deltamethrin yield improved residue profiles in dairy cattle milk, as compared to prior art formulations, thus enabling safe use of the inventive formulations in lactating dairy cows.

In another embodiment, the combination of a macrocyclic lactone and a synthetic pyrethroid delivered topically to cattle provides additional ectoparasiticide efficacy, as compared formulations comprising only a macrocyclic lactone. In some embodiments, the combination formulations provide synergistic efficacy against parasites, when compared to the additive efficacy of either active delivered separately.

In an embodiment, the instant invention provides a solution formulation in which both eprinomectin and deltamethrin are solubilised, for example, in MIGLYOL 840.

In another embodiment, the instant invention provides a suspension-type formulation in which eprinomectin is dissolved in a propylene glycol phase, while deltamethrin is suspended in an aqueous phase.

In an embodiment, the instant invention provides novel topical pyrethroid/macrocyclic lactone formulations with parasiticidal activity, for the treatment, prevention, reduction, or elimination of parasitic infestations and/or infection in an animal.

The instant invention further encompasses methods for the treatment or prevention of parasitic infestations and/or infection in animals, comprising the steps of administering an effective amount of formulations according to the instant invention to an animal in need thereof.

In an embodiment, the instant invention provides formulations containing eprinomectin as the macrocyclic lactone component, which formulations may exhibit shortened withdrawal times, thereby providing lactating mammals additional protection against pests and parasites. As compared to other known macrocyclic lactones, eprinomectin is understood to have the lowest milk to plasma partition ratio (˜0.2). For a point of reference and definition, a milk to plasma partition ratio of 1 would indicate the active tends to distribute evenly between milk and plasma.

In some embodiments, formulations described herein are particularly effective for controlling endoparasites, including, but in no way excluding, nematodes (such as roundworms, hookworms, whipworms and heartworms) and cestodes (tapeworms) and trematodes (flukes).

Therefore, the inventive formulations have substantial utility in preventing damage to crops, plants, plant propagation material and wood-containing property, and in controlling and preventing the infestation and/or infection of animals by parasites.

The invention includes at least the following features:

(a) In one embodiment, the invention provides novel topical formulations comprising pyrethroid and macrocyclic lactones, which are active against animal pests, including insects and parasites;

(b) methods for treating a parasitic infestation/infection in or on an animal are provided, which methods comprise administering a parasiticidally effective amount of a pyrethroid/macrocyclic lactone-containing formulation to the animal in need thereof;

(c) methods for the prevention of a parasitic infestation/infection of an animal, which comprise administering a parasiticidally effective amount the pyrethroid/macrocyclic lactone-containing formulation to the animal in need thereof;

(d) use of the formulations as a veterinary medicament for controlling pests, including parasites; and

(e) processes for the preparation of the pyrethroid/macrocyclic lactone-containing formulations.

Also contemplated within the scope of the invention are acid or base salts of the compounds in the compositions of the invention, where applicable.

The term “acid” contemplates all pharmaceutically acceptable inorganic or organic acids. Inorganic acids include mineral acids such as hydrohalic acids such as hydrobromic acid and hydrochloric acid, sulfuric acid, phosphoric acids and nitric acid. Organic acids include all pharmaceutically acceptable aliphatic, alicyclic and aromatic carboxylic acids, dicarboxylic acids, tricarboxylic acids and fatty acids. In one embodiment of the acids, the acids are straight chain or branched, saturated or unsaturated C₁-C₂₀ aliphatic carboxylic acids, which are optionally substituted by halogen or by hydroxyl groups, or C₆-C₁₂ aromatic carboxylic acids. Examples of such acids are carbonic acid, formic acid, acetic acid, propionic acid, isopropionic acid, valeric acid, α-hydroxy acids such as glycolic acid and lactic acid, chloroacetic acid, benzoic acid, methane sulfonic acid, and salicylic acid. Examples of dicarboxylic acids include oxalic acid, malic acid, succinic acid, tartaric acid, fumaric acid, and maleic acid. An example of a tricarboxylic acid is citric acid. Fatty acids include all pharmaceutically acceptable saturated or unsaturated aliphatic or aromatic carboxylic acids having 4 to 24 carbon atoms. Examples include butyric acid, isobutyric acid, sec-butyric acid, lauric acid, palmitic acid, stearic acid, oleic acid, linoleic acid, linolenic acid, and phenylsteric acid. Other acids include gluconic acid, glycoheptonic acid and lactobionic acid.

The term “base” contemplates all pharmaceutically acceptable inorganic or organic bases, including hydroxides, carbonates or bicarbonates of alkali metal or alkaline earth metals. Salts formed with such bases include, for example, the alkali metal and alkaline earth metal salts, including, but not limited to, as the lithium, sodium, potassium, magnesium or calcium salts. Salts formed with organic bases include the common hydrocarbon and heterocyclic amine salts, which include, for example, ammonium salts (NH₄⁺), alkyl- and dialkylammonium salts, and salts of cyclic amines such as the morpholine and piperidine salts.

In addition, the compounds within the compositions of the invention may exist as hydrates or solvates, in which a certain stoichiometric amount of water or a solvent is associated with the molecule in the crystalline form. The compositions of the invention may include hydrates and solvates of the active agents.

### DEFINITIONS

Terms used herein will have their customary meanings in the art unless specified. As used herein the term “anthelmintic” and variations thereof encompasses one or more nematocidal, trematocidal and cestocidal active compounds. The term “pesticidal” and variations thereof includes any said anthelmintic, any endectoparasiticidal, and/or any ectoparasiticidal compound. Where the context allows the term “ectoparasiticidal” includes compounds effective against any one or more ectoparasites including ticks, lice, fleas, mites and the like. Further, as used herein the “endectoparasiticidal” and variants thereof includes compounds and/or formulations that are active against internal (endo) and external (ecto) parasites.

As used herein the term “solubility” refers to the ability of a compound to be dissolved in a specific phase; and “lipophilic” means a greater tendency to an organic, oil or the like phase as opposed to another phase (for example, the aqueous phase).

As referred to herein the term “topical” refers to formulations that are applied topically, for example “pour-on” and “spot-on” formulations, or any topically-applied variants formulations thereof.

As referred to herein the term “acceptable storage stability” means stable for greater than 3 months at room temperature.

### Formulations of the Invention:

The formulations of the invention comprise at least pyrethroid and at least one macrocyclic lactone active compound, and may be applied topically to animals in need thereof, and are particularly effective at controlling/combating parasites, particularly ecto- and endoparasites. In certain embodiments, the formulations of the invention are useful in veterinary applications, including for controlling parasites in or on an animal.

In one embodiment, the at least one macrocyclic lactone active agent in the formulations of the invention may be at least one avermectin or milbemycin compound. In various embodiments, the at least one macrocyclic lactone compound may be abamectin, avermectin, dimadectin, doramectin, emamectin, eprinomectin, ivermectin, latidectin, lepimectin, selamectin, milbemectin, milbemycin D, milbemycin oxime, moxidectin or nemadectin.

In another embodiment, the at least one macrocyclic lactone active agent may be any one of the avermectins or avermectin monosaccharides modified in the 4′ or 4″ position described in U.S. Pat. No. 7,704,961, U.S. Pat. No. 7,521,429, US 2006-0105970 A1, U.S. Pat. No. 7,678,740, U.S. Pat. No. 7,632,820, U.S. Pat. No. 7,678,773, U.S. Pat. No. 7,605,134, and U.S. Pat. No. 6,933,260 (each to Merial). It will be appreciated by a skilled person that any of the above-recited avermectins exhibiting a relatively low milk to plasma partition ratio (for example less than about 1, less than about 0.5, and even more favorably, less than about 0.2) will be a particularly desirable component of formulations according to the instant invention.

In one embodiment the invention provides formulations according to the following:
(a) At least one synthetic pyrethroid;
(b) At least one macrocyclic lactone;
(c) At least one organic solvent;

In another embodiment the invention provides formulations according to the following:
(a) At least one synthetic pyrethroid;
(b) At least one macrocyclic lactone;
(c) At least one water miscible organic solvent;
(d) water.

In another embodiment, the invention provides a formulation comprising:
(a) At least one synthetic pyrethroid;
(b) At least one of ivermectin, abamectin, or eprinomectin, or a mixture thereof;
(c) At least one water miscible organic solvent;
(d) water.

In still another embodiment, the invention provides a formulation comprising:
(a) At least one synthetic pyrethroid;
(b) At least one of dimadectin, doramectin emamectin, latidectin, lepimectin or selamectin, or a mixture thereof;
(c) At least one water miscible organic solvent;
(d) water.

In yet another embodiment, the invention provides a formulation comprising:
(a) At least one synthetic pyrethroid;
(b) At least one of ivermectin, abamectin, or eprinomectin, or a mixture thereof;
(c) At least one water miscible organic solvent;
(d) water.

In another embodiment, the invention provides a formulation comprising:
(a) At least one synthetic pyrethroid;
(b) At least one of milbemectin, milbemycin D, milbemycin oxime, moxidectin or nemadectin, or a mixture thereof;
(c) At least one water miscible organic solvent;
(d) water.

In another embodiment, the invention provides a formulation comprising:
(a) Pyrethroid;
(b) At least one of ivermectin or eprinomectin, or a mixture thereof;
(c) At least one water miscible organic solvent;
(d) water.

In another embodiment, the invention provides a formulation comprising:
(a) Pyrethroid;
(b) At least one of avermectin or avermectin monosaccharide, each modified at the 4′ or 4″ position, or a mixture thereof;
(c) At least one water miscible organic solvent;
(d) water.

In an embodiment, the water miscible solvent will be an organic solvent, including an amide, alcohol, ester or sulfoxide. In another preferred embodiment, the formulations of the invention will comprise a pharmaceutically acceptable amide including, but not limited to, dimethylformamide, dimethylacetamide, 2-pyrrolidone, N-methylpyrrolidone and the like. In another embodiment, the solvent is MIGLYOL.

Surfactants are well known in the art and may include non-ionic surfactants, cationic surfactants and anionic surfactants. Anionic surfactants include, but are not limited to, alkaline stearates (e.g. sodium, potassium or ammonium stearate); calcium stearate or triethanolamine stearate; sodium abietate; alkyl sulfates, which include but are not limited to sodium lauryl sulfate and sodium cetyl sulfate; sodium dodecylbenzenesulphonate or sodium dioctyl sulphosuccinate; or fatty acids (e.g. coconut oil);

Cationic surfactants include, but are not limited to, any known water-soluble quaternary ammonium salts such as cetyltrimethylammonium bromide and the like; and known amine salts such as octadecylamine hydrochloride and the like.

Non-ionic surfactants include, but are not limited to, optionally polyoxyethylenated esters of sorbitan, e.g. POLYSORBATE 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide. Other surfactants that are suitable for the formulations of the present invention include amphoteric surfactants, such as substituted lauryl compounds of betaine. The surfactant of the inventive formulations may also be a mixture of at least two different surfactants.

The inventive formulations may contain other inert ingredients such as antioxidants, preservatives, or pH stabilizers. These compounds are well known in the formulation art. Antioxidant such as an alpha tocopherol, ascorbic acid, ascrobyl palmitate, fumaric acid, malic acid, sodium ascorbate, sodium metabisulfate, n-propyl gallate, BHA (butylated hydroxy anisole), BHT (butylated hydroxy toluene) monothioglycerol and the like, may be added to the present formulation. In certain embodiments, the antioxidants are generally added to the formulation in amounts of from about 0.01 to about 2.0%, based upon total weight of the formulation, with about 0.05 to about 1.0% being especially preferred.

In some embodiments, preservatives, such as the parabens (methylparaben and/or propylparaben), are suitably used in the formulation in amounts ranging from about 0.01 to about 2.0%, with about 0.05 to about 1.0% being especially preferred. Other preservatives include benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, and the like. Preferred ranges for these compounds include from about 0.01 to about 5%.

Compounds which stabilize the pH of the formulation are also contemplated and may be used in certain embodiments of the inventive formulations. Again, such compounds are well known to a practitioner in the art as well as how to use these compounds. Buffering systems include, for example, systems selected from the group consisting of acetic acid/acetate, malic acid/malate, citric acid/citrate, tataric acid/tartrate, lactic acid/lactate, phosphoric acid/phosphate, glycine/glycimate, tris, glutamic acid/glutamates and sodium carbonate.

### Parasites Controlled:

The topical pyrethroid/macrocyclic lactone formulations are particularly effective for efficiently controlling endoparasites, such as flukes, hookworms, and helminths such as cestodes, nematodes, and trematodes. Endoparasites further include helminths such as Anaplocephala, Ancylostoma, Anecator, Ascaris, Capillaria, Cooperia, Dipylidium, Dirofilaria, Echinococcus, Enterobius, Fasciola, Haemonchus, Oesophagostumum, Ostertagia, Toxocara, Strongyloides, Toxascaris, Trichinella, Trichuris, and Trichostrongylus. Or others from the class of helminths, such as from the class of helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

In one embodiment for treatment against ectoparasites, the ectoparasite is one or more insect or arachnid including those of the genera Ctenocephalides, Rhipicephalus, Dermacentor, Ixodes, Boophilus, Ambylomma, Haemaphysalis, Hyalomma, Sarcoptes, Psoroptes, Otodectes, Chorioptes, Hypoderma, Damalinia, Linognathus, Haematopinus, Solenoptes, Trichodectes, and Felicola.

In another embodiment for the treatment against ectoparasites, the ectoparasite is from the genera Ctenocephalides, Rhipicephalus, Dermacentor, Ixodes and/or Boophilus. The ectoparasites treated include but are not limited to fleas, ticks, mites, mosquitoes, flies, lice, blowfly and combinations thereof. Specific examples include but are not limited to cat and dog fleas (Ctenocephalides fells, Ctenocephalides sp. and the like), ticks (Rhipicephalus sp., Ixodes sp., Dermacentor sp., Amblyoma sp. and the like), and mites (Demodex sp., Sarcoptes sp., Otodectes sp. and the like), lice (Trichodectes sp., Cheyletiella sp., Lignonathus sp., and the like), mosquitoes (Aedes sp., Culex sp., Anopheles sp., and the like) and flies (Hematobia sp., Musca sp., Stomoxys sp., Dematobia sp., Cochliomyia sp., and the like). In yet another embodiment for the treatment against ectoparasites, the ectoparasite is a flea and/or tick.

Additional examples of ectoparasites include but are not limited to the tick genus Boophilus, especially those of the species microplus (cattle tick), decoloratus and annulatus; myiases such as Dermatobia hominis (known as Berne in Brazil) and Cochliomyia hominivorax (greenbottle); sheep myiases such as Lucilia sericata, Lucilia cuprina (known as blowfly strike in Australia, New Zealand and South Africa). Flies proper, namely those whose adult constitutes the parasite, such as Haematobia irritans (horn fly); lice such as Linognathus vitulorum, etc.; and mites such as Sarcoptes scabici and Psoroptes ovis. The above list is not exhaustive and other ectoparasites are well known in the art to be harmful to animals and humans. These include, for example migrating dipterous larvae.

When an anthelmintic agent is added to the composition of the invention, the composition can also be used to treat against endoparasites such as those helminths selected from the group consisting of Anaplocephala, Ancylostoma, Anecator, Ascaris, Capillaria, Cooperia, Dipylidium, Dirofilaria, Echinococcus, Enterobius, Fasciola, Haemonchus, Oesophagostumum, Ostertagia, Toxocara, Strongyloides, Toxascaris, Trichinella, Trichuris, and Trichostrongylus.

In another embodiment of the invention, the compounds and compositions of the invention are suitable for controlling pests such as insects selected from the group consisting of Blatella germanica, Heliothis virescens, Leptinotarsa decemlineata, Tetramorium caespitum and combinations thereof.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

In addition, with or without the other pesticidal agents added to the composition, the invention can also be used to treat other pests which include but are not limited to pests:

(1) from the order of Isopoda, for example Oniscus asellus, Armadillidium vulgare and Porcellio scaber;
(2) from the order of Diplopoda, for example Blaniulus guttulatus;
(3) from the order of Chilopoda, for example Geophilus carpophagus and Scutigera spp.;
(4) from the order of Symphyla, for example Scutigerella immaculate;
(5) from the order of Thysanura, for example Lepisma saccharina;
(6) from the order of Collembola, for example Onychiurus armatus;
(7) from the order of Blattaria, for example Blatta orientalis, Periplaneta americana, Leucophaea maderae and Blattella germanica;
(8) from the order of Hymenoptera, for example Diprion spp., Hoplocampa spp., Lasius spp., Monomorium pharaonis and Vespa spp.;
(9) from the order of Siphonaptera, for example Xenopsylla cheopis and Ceratophyllus spp.;
(10) from the order of Anoplura (Phthiraptera), for example, Damalinia spp., Haematopinus spp., Linognathus spp., Pediculus spp., Trichodectes spp.;
(11) from the class of Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.;
(12) from the class of Bivalva, for example, Dreissena spp.;
(13) from the order of Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.;
(14) from the order of Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella fit, Pegomyia hyoscyami, Phorbia spp., Stomoxys spp., Tabanus spp., Tannia spp., Tipula paludosa, Wohlfahrtia spp.;
(15) from the class of Gastropoda, for example, Anion spp., Biomphalaria spp., Bulinus spp., Deroceras spp., Galba spp., Lymnaea spp., Oncomelania spp., Succinea spp.;
(16) from the class of helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti;
(17) from the order of Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.;
(18) from the order of Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma pini, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona manginata, Carneocephala fulgida, Ceratovacuna lanigera, Cencopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus nibis, Dalbulus spp., Dialeunodes spp., Diaphorina spp., Diaspis spp., Dorsalis spp., Drosicha spp., Dysaphis spp., Dysmicoccus spp., Empoasca spp., Eniosoma spp., Erythnoneuna spp., Euscelis bilobatus, Geococcus coffeae, Homalodisca coagulata, Hyalopterus anundinis, Icenya spp., Idiocenus spp., Idioscopus spp., Laodelphax striatellus, Lecanium spp., Lepidosaphes spp., Lipaphis erysimi, Macnosiphum spp., Mahananva fimbriolata, Melanaphis sacchani, Metcalfiella spp., Metopolophium dinhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.;
(19) from the order of Isoptera, for example, Reticulitermes spp., Odontotermes spp.;
(20) from the order of Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.;
(21) from the order of Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.;
(22) from the order of Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.;
(23) from the class of Protozoa, for example, Eimeria spp.

### EXAMPLES

The following examples are provided to illustrate certain embodiments of the invention and are not to be construed in any way as limiting the scope of the invention.

### Example 1

### Pour-on Suspension Formulations Comprising Eprinomectin and Deltamethrin

TABLE 1
Suspension formulations

| | Formulation | | | | |
|---|---|---|---|---|---|
| Batch No | | % w/v | Purpose, method of manufacture and comments | | |
| SOW 74/75-03 | Eprinomectin | 1.00 | Purpose: Preliminary screening trial to determine a suitable | | |
| (70% | Deltamethrin | 1.50 | base for Eprinomectin and Deltamethrin PO (Suspension) | | |
| Propylene | Methyl Paraben Sodium | 0.18 | Result: Stress test results indicate both actives are stable | | |
| Glycol: q.s to | Propyl Paraben Sodium | 0.02 | | 70° C. 5 days | |
| Water) - 1% | BHT | 0.03 | | Assay compared to initial | |
| Eprinomectin | Propylene Glycol (Part1) | 70.0 | B. No. | Eprinomectin | Deltamethrin |
| & 1.5% | Defoamer | 2.00 | 03 | 100.7% | 98.3% |
| Deltamethrin | Aerosil 200 | 0.30 | | | |
| | Xanthan Gum | 0.30 | | | |
| | Propylene Glycol(Part 2) | 0.60 | | | |
| | Purified Water | to 100 | | | |
| | Citric Acid Anhydrous | Q.s for pH | | | |
| SOW 74/75-04 | Eprinomectin | 0.50 | Purpose: Batch prepared with lower concentration for | | |
| (70% | Deltamethrin | 0.75 | Eprinomectin & Deltamethrin | | |
| Propylene | Methyl Paraben Sodium | 0.18 | Result: Stress test results | | |
| Glycol: q.s to | Propyl Paraben Sodium | 0.02 | | 70° C. 5 days | |
| Water) - 0.5% | Citric Acid Anhydrous | 0.10 | | Assay compared to initial | |
| Eprinomectin | BHT | 0.03 | B. No. | Eprinomectin | Deltamethrin |
| & 0.75% | Propylene Glycol (Part1) | 70.0 | 04 | 101.0% | 68.4% |
| Deltamethrin | Defoamer | 1.00 | | | |
| | Aerosil 200 | 0.30 | | | |
| | Xanthan Gum | 0.30 | | | |
| | Propylene Glycol(Part 2) | 0.60 | | | |
| | Titanium Dioxide 3328 | 0.10 | | | |
| | FD & C Yellow no5 lake | 0.03 | | | |
| | Purified Water | to 100 | | | |
| SOW 74/75-05 | Eprinomectin | 0.50 | Purpose: Batch prepared with lower concentration of | | |
| (60% | Deltamethrin | 0.75 | Propylene Glycol & Dye | | |
| Propylene | Methyl Paraben Sodium | 0.18 | Result: Stress test results | | |
| Glycol: q.s to | Propyl Paraben Sodium | 0.02 | | 70° C. 5 days | |
| Water) - 0.5% | Citric Acid Anhydrous | 0.10 | | Assay compared to initial | |
| Eprinomectin | BHT | 0.03 | B. No. | Eprinomectin | Deltamethrin |
| & 0.75% | Propylene Glycol (Part1) | 60.0 | 05 | 101.7% | 92.0% |
| Deltamethrin | Defoamer | 1.00 | | | |
| | Aerosil 200 | 0.30 | | | |
| | Xanthan Gum | 0.30 | | | |
| | Propylene Glycol(Part 2) | 0.60 | | | |
| | Titanium Dioxide 3328 | 0.10 | | | |
| | FD & C Yellow no5 lake | 0.03 | | | |
| | Purified Water | to 100 | | | |
| SOW 74/75-06 | Eprinomectin | 0.50 | Purpose: Batch prepared with 70% Propylene Glycol & New | | |
| (70% | Deltamethrin | 0.75 | Eprinomectin API | | |
| Propylene | Methyl Paraben Sodium | 0.18 | Result: Stress test results | | |
| Glycol: q.s to | Propyl Paraben Sodium | 0.02 | | 70° C. 5 days | |
| Water) - 0.5% | Citric Acid Anhydrous | 0.10 | | Assay compared to initial | |
| Eprinomectin | BHT | 0.03 | B. No. | Eprinomectin | Deltamethrin |
| & 0.75% | Propylene Glycol (Part1) | 70.0 | 06 | 101.6% | 65.1% |
| Deltamethrin | Defoamer | 1.00 | | | |
| | Aerosil 200 | 0.30 | | | |
| | Xanthan Gum | 0.30 | | | |
| | Propylene Glycol(Part 2) | 0.60 | | | |
| | Titanium Dioxide 3328 | 0.10 | | | |
| | FD & C Yellow no5 lake | 0.03 | | | |
| | Purified Water | to 100 | | | |
| SOW 74/75-07 | Eprinomectin | 0.50 | Purpose: Batch prepared with lower concentration of | | |
| (60% | Deltamethrin | 0.75 | Propylene Glycol (60%) & New Eprinomectin API | | |
| Propylene | Methyl Paraben Sodium | 0.18 | Result: Stress test results | | |
| Glycol: q.s to | Propyl Paraben Sodium | 0.02 | | 70° C. 5 days | |
| Water) - 0.5% | Citric Acid Anhydrous | 0.10 | | Assay compared to initial | |
| Eprinomectin | BHT | 0.03 | B. No. | Eprinomectin | Deltamethrin |
| & 0.75% | Propylene Glycol (Part1) | 60.0 | 07 | 101.0% | 89.5% |
| Deltamethrin | Defoamer | 1.00 | | | |
| | Aerosil 200 | 0.30 | | | |
| | Xanthan Gum | 0.30 | | | |
| | Propylene Glycol(Part 2) | 0.60 | | | |
| | Titanium Dioxide 3328 | 0.10 | | | |
| | FD & C Yellow no5 lake | 0.03 | | | |
| | Purified Water | to 100 | | | |
| SOW 74/75-08 | Eprinomectin | 0.50 | Purpose: Batch prepared with lower concentration of | | |
| (60% | Deltamethrin | 0.75 | Propylene Glycol (60%) & New Eprinomectin API | | |
| Propylene | Methyl Paraben Sodium | 0.18 | Result: Stress test results | | |
| Glycol: q.s to | Propyl Paraben Sodium | 0.02 | | 70° C./5 | 55° C./1 |
| Water) - 0.5% | Citric Acid Anhydrous | 0.10 | B. No.: 08 | days | month |
| Eprinomectin | BHT | 0.03 | Eprinomectin | 98.4% | 95.1% |
| & 0.75% | Propylene Glycol (Part1) | 60.0 | Deltamethrin | 85.6% | 96.7% |
| Deltamethrin | Defoamer | 1.00 | *Assay compared to initial | | |
| | Aerosil 200 | 0.30 | | | |
| | Xanthan Gum | 0.30 | | | |
| | Propylene Glycol(Part 2) | 0.60 | | | |
| | Titanium Dioxide 3328 | 0.10 | | | |
| | FD & C Yellow no5 lake | 0.03 | | | |
| | Purified Water | to 100 | | | |
| SOW 74/75-09 | Eprinomectin | 0.50 | Purpose: Batch prepared with lower concentration of | | |
| (57% | Deltamethrin | 0.75 | Propylene Glycol (57%) & New Eprinomectin API | | |
| Propylene | Methyl Paraben Sodium | 0.18 | Result: Stress test results | | |
| Glycol: q.s to | Propyl Paraben Sodium | 0.02 | | 70° C./5 | 55° C./1 |
| Water) - 0.5% | Citric Acid Anhydrous | 0.10 | B. No.: 09 | days | month |
| Eprinomectin | BHT | 0.03 | Eprinomectin | 100.2% | 100.8% |
| & 0.75% | Propylene Glycol (Part1) | 57.0 | Deltamethrin | 84.5% | 98.6% |
| Deltamethrin | Defoamer | 1.00 | *Assay compared to initial | | |
| | Aerosil 200 | 0.30 | | | |
| | Xanthan Gum | 0.30 | | | |
| | Propylene Glycol(Part 2) | 0.60 | | | |
| | Titanium Dioxide 3328 | 0.10 | | | |
| | FD & C Yellow no5 lake | 0.03 | | | |
| | Purified Water | to 100 | | | |
| SOW 74/75-10 | Eprinomectin | 0.50 | Purpose: Batch prepared with lower concentration of | | |
| (55% | Deltamethrin | 0.75 | Propylene Glycol (55%) & New Eprinomectin API | | |
| Propylene | Methyl Paraben Sodium | 0.18 | Result: Stress test results | | |
| Glycol: q.s to | Propyl Paraben Sodium | 0.02 | | 70° C./5 | 55° C./1 |
| Water) - 0.5% | Citric Acid Anhydrous | 0.10 | B. No.: 10 | days | month |
| Eprinomectin | BHT | 0.03 | Eprinomectin | 100.0% | 101.0% |
| & 0.75% | Propylene Glycol (Part1) | 55.0 | Deltamethrin | 95.9% | 100.0% |
| Deltamethrin | Defoamer | 1.00 | *Assay compared to initial | | |
| | Aerosil 200 | 0.30 | | | |
| | Xanthan Gum | 0.30 | | | |
| | Propylene Glycol(Part 2) | 0.60 | | | |
| | Titanium Dioxide 3328 | 0.10 | | | |
| | FD & C Yellow no5 lake | 0.03 | | | |
| | Purified Water | to 100 | | | |
| SOW 74/75-11 | Eprinomectin | 0.50 | Purpose: Batch prepared with 30% purified water & q.s to | | |
| (30% Water: | Deltamethrin | 0.75 | Propylene Glycol | | |
| q.s to | Methyl Paraben Sodium | 0.18 | Result: Stress test results | | |
| Propylene | Propyl Paraben Sodium | 0.02 | | 70° C./5 | 55° C./1 |
| Glycol) - 0.5% | Citric Acid Anhydrous | 0.10 | B. No.: 11 | days | month |
| Eprinomectin | BHT | 0.03 | Eprinomectin | 98.3% | 99.0% |
| & 0.75% | Propylene Glycol (Part1) | to 100 | Deltamethrin | 22.1% | 71.6% |
| Deltamethrin | Defoamer | 1.00 | *Assay compared to initial | | |
| | Aerosil 200 | 0.30 | | | |
| | Xanthan Gum | 0.30 | | | |
| | Propylene Glycol(Part 2) | 0.60 | | | |
| | Titanium Dioxide 3328 | 0.10 | | | |
| | FD & C Yellow no5 lake | 0.03 | | | |
| | Purified Water | 30.0 | | | |
| SOW 74/75-14 | Eprinomectin | 0.50 | Purpose: Batch prepared with 35% purified water & q.s to | | |
| (35% Water: | Deltamethrin | 0.75 | Propylene Glycol | | |
| q.s to | Methyl Paraben Sodium | 0.18 | Result: Stress test results | | |
| Propylene | Propyl Paraben Sodium | 0.02 | | 70° C./5 | 55° C./1 |
| Glycol) - 0.5% | Citric Acid Anhydrous | 0.10 | B. No.: 14 | days | month |
| Eprinomectin | BHT | 0.03 | Eprinomectin | 100.8% | 101.2% |
| & 0.75% | Propylene Glycol (Part1) | to 100 | Deltamethrin | 67.7% | 98.4% |
| Deltamethrin | Defoamer | 1.00 | *Assay compared to initial | | |
| | Aerosil 200 | 0.30 | | | |
| | Xanthan Gum | 0.30 | | | |
| | Propylene Glycol(Part 2) | 0.60 | | | |
| | Titanium Dioxide 3328 | 0.10 | | | |
| | FD & C Yellow no5 lake | 0.03 | | | |
| | Purified Water | 35.0 | | | |
| SOW 74/75-15 | Eprinomectin | 0.50 | Purpose: Batch prepared with 40% purified water & q.s to | | |
| (40% Water: | Deltamethrin | 0.75 | Propylene Glycol | | |
| q.s to | Methyl Paraben Sodium | 0.18 | Result: Stress test results | | |
| Propylene | Propyl Paraben Sodium | 0.02 | | 70° C./5 | 55° C./1 |
| Glycol) - 0.5% | Citric Acid Anhydrous | 0.10 | B. No.: 15 | days | month |
| Eprinomectin | BHT | 0.03 | Eprinomectin | 102.2% | 99.0% |
| & 0.75% | Propylene Glycol (Part1) | to 100 | Deltamethrin | 67.1% | 96.0% |
| Deltamethrin | Defoamer | 1.00 | *Assay compared to initial | | |
| | Aerosil 200 | 0.30 | | | |
| | Xanthan Gum | 0.30 | | | |
| | Propylene Glycol(Part 2) | 0.60 | | | |
| | Titanium Dioxide 3328 | 0.10 | | | |
| | FD & C Yellow no5 lake | 0.03 | | | |
| | Purified Water | 40.0 | | | |
| SOW 74/75-16 | Eprinomectin | 0.50 | Purpose: Batch prepared with 43% purified water & q.s to | | |
| (43% Water: | Deltamethrin | 0.75 | Propylene Glycol. Add surfactant. | | |
| q.s to | Methyl Paraben Sodium | 0.18 | Result: Stress test results | | |
| Propylene | Propyl Paraben Sodium | 0.02 | | 70° C./5 | 55° C./1 |
| Glycol) - 0.5% | Citric Acid Anhydrous | 0.10 | B. No.: 16 | days | month |
| Eprinomectin | BHT | 0.03 | Eprinomectin | 99.2% | 99.8% |
| & 0.75% | Propylene Glycol (Part1) | to 100 | Deltamethrin | 100.0% | 95.8% |
| Deltamethrin | Polysorbate 80 | 0.15 | *Assay compared to initial | | |
| | Defoamer | 1.00 | | | |
| | Aerosil 200 | 0.30 | | | |
| | Xanthan Gum | 0.15 | | | |
| | Propylene Glycol(Part 2) | 0.30 | | | |
| | Titanium Dioxide 3328 | 0.10 | | | |
| | FD & C Yellow no5 lake | 0.01 | | | |
| | Purified Water | 43.0 | | | |
| SOW 74/75-17 | Eprinomectin | 0.50 | Purpose: Batch prepared with 45% purified water & q.s to | | |
| (45% Water: | Deltamethrin | 0.75 | Propylene Glycol | | |
| q.s to | Methyl Paraben Sodium | 0.18 | Result: Stress test results | | |
| Propylene | Propyl Paraben Sodium | 0.02 | | 70° C./5 | 55° C./1 |
| Glycol) - 0.5% | Citric Acid Anhydrous | 0.10 | B. No.: 17 | days | month |
| Eprinomectin | BHT | 0.03 | Eprinomectin | 99.4% | 96.7% |
| & 0.75% | Propylene Glycol (Part1) | to 100 | Deltamethrin | 98.5% | 97.1% |
| Deltamethrin | Polysorbate 80 | 0.15 | *Assay compared to initial | | |
| | Defoamer | 1.00 | | | |
| | Aerosil 200 | 0.30 | | | |
| | Xanthan Gum | 0.30 | | | |
| | Propylene Glycol(Part 2) | 0.15 | | | |
| | Titanium Dioxide 3328 | 0.10 | | | |
| | FD & C Yellow no5 lake | 0.01 | | | |
| | Purified Water | 45.0 | | | |
| SOW 74/75-18 | Eprinomectin | 0.50 | Purpose: Batch prepared with 55% Propylene Glycol & q.s to | | |
| (55% | Deltamethrin | 0.75 | Purified water (Batch submit for clinical trial). Reduce | | |
| Propylene | Methyl Paraben Sodium | 0.18 | Xanthan gum concentration. | | |
| Glycol: q.s to | Propyl Paraben Sodium | 0.02 | Result: Stress test results | | |
| Water) - 0.5% | Citric Acid Anhydrous | 0.10 | | 70° C./5 | 55° C./1 |
| Eprinomectin | BHT | 0.03 | B. No.: 18 | days | month |
| & 0.75% | Propylene Glycol (Part1) | 55.0 | Eprinomectin | 98.2% | 102.4% |
| Deltamethrin | Polysorbate 80 | 0.15 | Deltamethrin | 100.0% | 98.9% |
| | Defoamer | 1.00 | *Assay compared to initial | | |
| | Aerosil 200 | 0.30 | | | |
| | Xanthan Gum | 0.15 | | | |
| | Propylene Glycol(Part 2) | 0.30 | | | |
| | Titanium Dioxide 3328 | 0.10 | | | |
| | FD & C Yellow no5 lake | 0.01 | | | |
| | Purified Water | to 100 | | | |
| SOW 74/75-20 | Eprinomectin | 0.50 | Purpose: Batch prepared for clinical study | | |
| Batch for large | Deltamethrin | 0.75 | | | |
| site reaction | Methyl Paraben Sodium | 0.18 | | | |
| study | Propyl Paraben Sodium | 0.02 | | | |
| | Citric Acid Anhydrous | 0.10 | | | |
| | BHT | 0.03 | | | |
| | Propylene Glycol (Part1) | 55.0 | | | |
| | Polysorbate 80 | 0.15 | | | |
| | Defoamer | 1.00 | | | |
| | Aerosil 200 | 0.30 | | | |
| | Xanthan Gum | 0.15 | | | |
| | Propylene Glycol(Part 2) | 0.30 | | | |
| | Titanium Dioxide 3328 | 0.10 | | | |
| | FD & C Yellow no5 lake | 0.01 | | | |
| | Purified Water | to 100 | | | |
| SOW 74/75-22 | Eprinomectin | 0.50 | 2000 mL batch for clinical study | | |
| | Deltamethrin | 0.75 | | | |
| | Methyl Paraben Sodium | 0.18 | | | |
| | Propyl Paraben Sodium | 0.02 | | | |
| | Citric Acid Anhydrous | 0.10 | | | |
| | BHT | 0.03 | | | |
| | Propylene Glycol (Part1) | 55.0 | | | |
| | Polysorbate 80 | 0.15 | | | |
| | Defoamer | 1.00 | | | |
| | Aerosil 200 | 0.30 | | | |
| | Xanthan Gum | 0.15 | | | |
| | Propylene Glycol (Part 2) | 0.30 | | | |
| | Titanium Dioxide 3328 | 0.10 | | | |
| | FD & C Yellow no5 lake | 0.01 | | | |
| | Purified Water | to 100 | | | |

### Example 2

### Pour-on Solution Formulations Comprising Eprinomectin and Deltamethrin

TABLE 2
Solution formulations

| | Formulation | | | | |
|---|---|---|---|---|---|
| Batch No | | % w/v | Purpose, method of manufacture and comments | | |
| SOW 74/75- | Eprinomectin | 1.00 | Purpose: Preliminary trial by adding 0.5% Eprinomectin | | |
| Trial 01 | Deltamethrin | 1.50 | & 1.5% Deltamethrin into marketed product. | | |
| 1% | Eprinex PO | to 100 | Result: Stress test results indicate both actives are stable | | |
| Eprinomectin | (Marketed Product | | | 70° C. 5 days | |
| & 1.5% | Batch No.: 802483) | | | Assay compared to initial | |
| Deltamethrin | | | B. No. | Eprinomectin | Deltamethrin |
| | | | Trial 01 | 100.0% | 99.3% |
| SOW 74/75- 19 | Eprinomectin | 0.50 | Purpose: Batch prepared by adding 0.75% Deltamethrin | | |
| 0.5% | Deltamethrin | 0.75 | into marketed product. | | |
| Eprinomectin | Eprinex PO | to 100 | Result: Stress test results indicate both actives are stable | | |
| & 0.75% | (Marketed Product | | | 70° C. 5 days | |
| Deltamethrin | Batch No.: 802483) | | | Assay compared to initial | |
| | | | B. No. | Eprinomectin | Deltamethrin |
| | | | 19 | 100.7% | 98.9% |
| SOW 74/75- 21 | Eprinomectin | 0.50 | Purpose: Batch prepared for clinical study | | |
| Batch for large | Deltamethrin | 0.75 | | | |
| site reaction | Eprinex PO | to 100 | | | |
| study | (Marketed Product | | | | |
| | Batch No.: 802483) | | | | |
| SOW 74/75- 23 | Eprinomectin | 0.50 | 1000 mL | | |
| Batch prepared | Deltamethrin | 0.75 | | | |
| for clinical | Miglyol 840 | to 100 | | | |
| study | | | | | |

### Example 3

### Formulations Advanced to Clinical Assessment

Based on the development work, two formulations were selected for clinical assessment (summarized in Tables 3-5).

TABLE 3
Formulation of Eprinomectin/Deltamethrin Pour On (Suspension)

| | Composition | |
|---|---|---|
| Ingredient | (% w/v) | Function |
| Methyl Paraben Sodium | 0.18 | Preservative |
| Propyl Paraben Sodium | 0.02 | Preservative |
| Citric Acid anhydrous | 0.10 | Buffer |
| Deltamethrin¹ | 0.75 | Active |
| POLYSORBATE 80 | 0.15 | Surfactant |
| Defoamer | 1.00 | Anti foaming agent |
| Eprinomectin¹ | 0.50 | Active |
| BHT | 0.03 | Anti oxidant |
| Propylene Glycol | 55.00 | Vehicle |
| Aerosil 200 | 0.30 | Anti caking agent |
| Xanthan Gum | 0.15 | Suspending Agent |
| Propylene Glycol | 0.30 | Dispersing Agent |
| Titanium Dioxide 3328 | 0.10 | Opacifier |
| FD &C Yellow no. 5 alum lake | 0.01 | Colouring agent |
| Deionised Water | q.s to 100 | Vehicle |

¹Added after potency correction

TABLE 4
Formulation Manufacturing Procedure

| | |
|---|---|
| 1. | Dispense Propylene Glycol in a vessel. |
| 2. | Add Eprinomectin and BHT to step 1 with mixing, mix till clear solution achieved. |
| | Preparation of Preservative Solution |
| (a) | Dispense Purified water (30% of batch volume) in a vessel. |
| (b) | Add Methyl Paraben Sodium and Propyl Paraben Sodium to step (a) with mixing, mix till clear solution achieved. |
| 3. | To step 2 add preservative solution. Rinse the container with Purified water and add to step 2. Mix with |
| | homogenization. |
| 4. | Add Citric Acid Anhydrous and Polysorbate 80 to step 3 with mixing, mix till clear solution achieved. |
| 5. | Add and disperse Defoamer with homogenization. |
| 6. | Add and disperse Deltamethrin. Mix with homogenization for 30 minutes. |
| 7. | To the bulk add Aerosil 200. Mix with homogenization. |
| 8. | In a separate mixing vessel add Propylene Glycol and disperse Xanthan Gum. Allow to wet for 2 hours. |
| 9. | Add the dispersion from step 7 to the bulk. |
| 10. | Rinse the container from step 9 with Purified water. Add the rinsing to the bulk mixing vessel. |
| 11. | Mix with homogenization for 15 minutes. |
| 12. | Withdraw 10 g of sample in a glass beaker. Check for presence of large particles/lumps. |
| | If large particles/lumps are observed, continue mixing and homogenization for further 30 minutes. |
| | If no large particles/lumps are seen, proceed to step 13. |
| 13. | Add titanium dioxide with stirring. |
| 14. | Add FD & C yellow no. 5 alum lake in 100 mL purified water separately. Add this suspension to step 13. |
| 15. | Check the pH of the dispersion. Limits: 5.0-7.0 |
| 16. | Make up the volume with Purified water |
| 17. | Mix with homogenization for 30 minutes |

TABLE 5
Formulation of Eprinomectin/Deltamethrin Pour On (Solution)

| Ingredient | Composition (% w/v) | Function |
|---|---|---|
| Eprinomectin¹ | 0.50 | Active |
| Deltamethrin¹ | 0.75 | Active |
| Miglyol 840 | q.s to 100 | Vehicle |

¹To be added after potency correction

The manufacturing procedure for the solution was carried out according to the following:

1) loaded 95% MIGLYOL 840 & eprinomectin;

2) added deltamethrin with mixing until the solution was clear;

3) made to volume with MIGLYOL 840.

### Example 4

### Clinical Studies

Studies were performed with both formulations in comparison with registered products to examine critical parameters: pour-on application site safety, milk residue levels of both active ingredients, plasma concentration of eprinomectin. These studies were done in a step wise fashion to determine if one formulation was superior to the other. The safety study was performed first as a pour-on cannot proceed through further development if it causes administration site reactions. The pilot milk residue study was performed next as a critical element in the marketing of this pour-on is that it has a nil milk with-holding period. Lastly the Eprinomectin plasma study was perform to demonstrate that the product absorbs sufficiently to be efficacious against internal parasites. It was especially important to evaluate sensitivity, as is well known in the art that synthetic pyrethroid (SP) solution-based formulations are more likely to result in SP sensitivity reactions than suspension-based SP formulations.

### Study 1—Pour-on Site Safety and Irritability

Study objective was to determine whether a site reaction occurred in dairy cattle when topical applications of eprinomectin/deltamethrin pour-on were administered on two occasions, with a two week interval between treatments. Two different formulations were tested.

Methods.

Twenty Jersey cows were ear tagged, weighed and then randomly allocated into two groups of 10. Animals in Group 1 were treated with IVP 1 (Eprinomectin/Deltamethrin solution) on day 0 and again on day 14. Animals in Group 2 were treated with IVP 2 (Eprinomectin/Deltamethrin suspension) on day 0 and again on day 14. All animals were observed for adverse reaction at 30 mins, and 1, 2 and 6 hours post each treatment event and on selected days out to 48 days post the first treatment. The following parameters were assessed at the pour-on site: swelling, erythema, skin flaking, alopecia. Animals were also observed for general behavior post-treatment with attention to any licking of the pour-on site.

Results.

No site reactions of any kind manifested in any animal following treatment with either IVP 1 or IVP 2 at any of the observation time points. One animal, showed possible discomfort after the repeat treatment on Day 14 with IVP 1 (solution). Oral behaviour was observed, with several attempts to lick at the treated area. This behaviour was not observed at any of the subsequent observation time points. Therefore, both formulations were considered safe and were put forward for testing in the pilot milk residue study. A larger study may be performed to conclusively demonstrate lack of SP sensitivity reactions, though initial results were encouraging.

### Study 2—Milk Residues

This study was performed to establish whether a nil milk withholding period was likely after a single topical treatment with one of two experimental eprinomectin/deltamethrin pour on formulations. The study was a single dose milk residue pilot study. Two positive control groups were included for comparison.

Methods. Seventeen lactating dairy cows were selected for the study. One animal was selected as the untreated control and the remaining animals were allocated into 4 groups of 4 on milk yield. Treatments were allocated according to Table 6.

TABLE 6
Treatments

| Group | n | Product | Dose | Route |
|---|---|---|---|---|
| 1 | 4 | Eprinomectin/deltamethrin | 1 mL/10 kg | Topical |
| | | Formulation 22 (suspension) | | |
| 2 | 4 | Eprinomectin/deltamethrin | 1 mL/10 kg | Topical |
| | | Formulation 23 (solution) | | |
| 3 | 4 | STAMPEDE Easy Dose | 1 mL/20 kg | Topical |
| 4 | 4 | Ivomec EPRINEX | 1 mL/10 kg | Topical |
| 5 | 1 | Negative Control | NA | NA |

On Day 0, the cows were weighed and milk samples were collected from each animal at the afternoon milking prior to treatment. After the milking, each animal was treated with the relevant formulation at the appropriate dose rate, calculated on bodyweight and rounded up to the nearest 1 mL. The formulations were applied using a different disposable syringe for each product; they were administered by topical application in a narrow strip along the dorsal midline from the withers to the tailhead. After treatment, De Laval herd test equipment was used to collect individual, representative milk samples from each study animal at each milking until the morning milking on Day 8. Samples were stored frozen at or colder than −18° C. Primary samples were couriered to the analytical laboratory in frozen state and assayed for the relevant residues using validated instrumental methods.

Results.

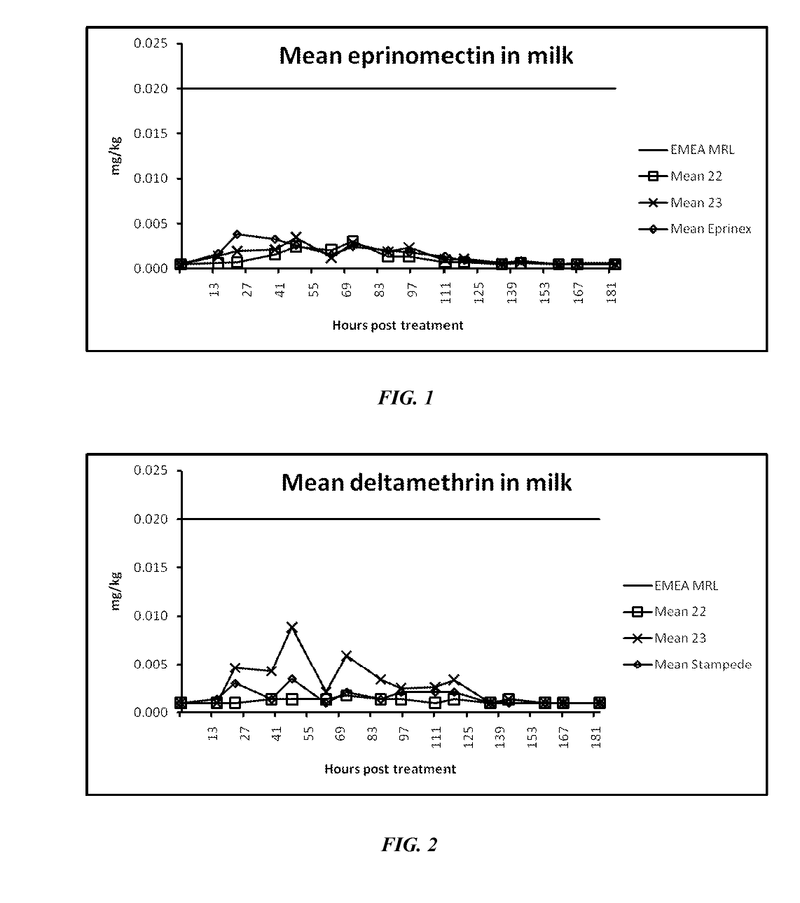

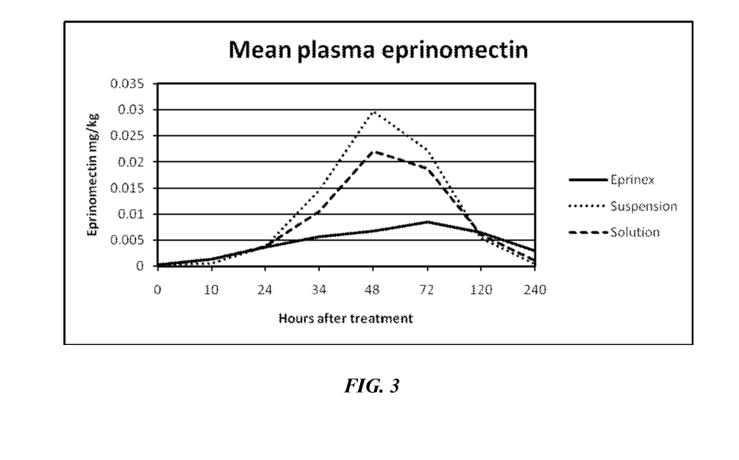

Group mean Eprinomectin and Deltamethrin concentrations at each milking after treatment are presented in FIGS. 1 and 2 respectively. The EMEA MRL's for both actives are presented to provide reference points. FIG. 3 presents mean plasma concentration of eprinomectin at time points post-administration of EPRINEX, the inventive suspension, and the inventive solution. Both formulations are expected to have nil milk with-holding periods. Based on the results of this study both formulations were taken through to the Eprinomectin plasma concentration study. No animals had deltamethrin or eprinomectin milk residues above the MRL at any time point during the study.

### Study 3—Determination of Eprinomectin Plasma Concentrations

This study aimed to determine the plasma concentration-time curves for eprinomectin in cattle after a single treatment with one of two combination eprinomectin/deltamethrin pour on formulations. The eprinomectin plasma concentration-time curves for the Investigational Veterinary Products were compared to a registered product (EPRINEX® Pour On) to determine whether the rate and extent of absorption of eprinomectin was sufficient to achieve anthelmintic efficacy. This was a randomised controlled pharmacokinetic pilot study.

Methods.

On Day −1, the study animals were blood sampled, weighed, ranked by body weight and restrictively randomly allocated into three groups of six on the basis of body weight.

On Day 0, groups were randomly allocated to one of the three treatments using the draw-from the-hat method. Animals in Group 1 were treated with EPRINEX® Pour On. Animals in Group 2 were treated with IVP Formulation 2: Eprinomectin/deltamethrin solution. Animals in Group 3 were treated with IVP Formulation 1: Eprinomectin/deltamethrin suspension. Individual doses were calculated at 1 mL/10 kg (0.5 mg eprinomectin/kg) based on Day −1 bodyweight. Calculated doses were rounded up to the nearest minor increment of the applicator (1 mL) for administration. All formulations were administered by topical application in a strip along the dorsal midline between the withers and the tailhead using a new “non-splatter” applicator for each product (Merial Ancare Simcro applicator for pour-on).

Animals were treated by group and were kept separated by group after treatments were administered. They were protected from rain in group pens in a covered facility until after the Day 5 (120 hours) bleed. Following treatment, all study animals were blood sampled at 10, 24, 34, 48, 72, 120 and 240 hours post treatment. Blood samples were collected by jugular venipuncture into duplicate heparinised vacutainers and were immediately centrifuged and the plasma was decanted into duplicate 5 mL tubes. Plasma samples were stored in a freezer and the appropriate samples were submitted to the analytical lab at the end of the study. Samples were analysed for eprinomectin B1a using a validated instrumental method with an LOD and LOQ of 0.0005 and 0.001 mg/kg respectively.

Results.

FIG. 3 shows the group mean plasma eprinomectin concentration time curves for the three formulations tested in the study. Both developmental Eprinomectin/Deltamethrin formulations produce higher peak concentrations of Eprinomectin than EPRINEX pour-on. The two developmental Eprinomectin/Deltamethrin pour-on formulations tested in this study appeared to absorb more effectively than EPRINEX Pour-On. As a result both developmental pour-on formulations showed higher mean plasma Eprinomectin levels after treatment. It is concluded from this study that both Eprinomectin/Deltamethrin formulations would be at least as efficacious against internal parasites as EPRINEX Pour-On.

### Study 4—Efficacy of a Combination Eprinomectin and Deltamethrin Pour on Suspension Against Biting Lice (Bovicola Bovis) and Sucking Lice in Cattle

This study was a randomised efficacy study in naturally infected animals, with negative and positive treatment groups. A group of more than twenty animals suspected of harboring louse infestation (on the basis of visible hair loss around the neck, withers and/or rump) were located. A selection of louse infected animals was then confirmed (by gross identification) and lice numbers. The animals were identified with uniquely numbered ear tags.

Methods.

On Day 0, individual total lice counts were performed. The twenty animals with the highest total lice counts were selected for the study and restrictively randomised into two treatment groups of ten animals each, based on total lice count. On Day 0, all study animals were weighed and Groups 1 treated with the Eprinomectin/deltamethrin Pour-On treatment. Group 2 remained untreated. Each group was then run in a separate enclosure after treatment. Individual lice counts were subsequently performed on each study animal on Day 7, 14, 28, 42 and 56.

Results.

The result demonstrated that the Eprinomectin/deltamethrin suspension based formulation was highly effective at controlling Biting Lice (Bovicloa bovis) throughout the 56 day study period.

TABLE 7
Treatment details

| Tag 1 | Tag 2 | Tx | D 0 | D 7 | D 14 | D 31 | D 42 | D 56 |
|---|---|---|---|---|---|---|---|---|
| 7 | 46 | Eprino/Delta | 106 | 0 | 0 | 0 | 0 | |
| 45 | 23 | Eprino/Delta | 171 | 0 | 0 | 0 | 0 | |
| 81 | 38 | Eprino/Delta | 66 | 0 | 0 | 0 | 0 | |
| 171 | 36 | Eprino/Delta | 42 | 0 | 0 | 0 | 0 | |
| 295 | 38 | Eprino/Delta | 92 | 0 | 0 | 0 | 0 | |
| 1392 | Blank | Eprino/Delta | 49 | 0 | 0 | 0 | 0 | |
| | Green | | | | | | | |
| 1401 | 31 | Eprino/Delta | 129 | 0 | 0 | 0 | 0 | |
| 1478 | 43 | Eprino/Delta | 54 | 0 | 0 | 0 | 0 | |
| 133 | 24 | Negative | 171 | 135 | 153 | 77 | 25 | |
| 169 | 22 | Negative | 62 | 45 | 43 | 61 | 50 | |
| 299 | 50 | Negative | 270 | 213 | 189 | 127 | 92 | |
| 473 | 37 | Negative | 52 | 50 | 33 | 77 | 31 | |
| 1447 | 45 | Negative | 128 | 89 | 74 | 116 | 47 | |
| 1444 | 34 | Negative | 31 | 40 | 57 | 61 | 39 | |
| 1492 | 35 | Negative | 94 | 119 | 124 | 105 | 139 | |
| 3122 | 42 | Negative | 72 | 44 | 26 | 57 | 36 | |

TABLE 8
Arithmetic means

| | D 0 | D 7 | D 14 | D 31 | D 42 | D 56 |
|---|---|---|---|---|---|---|
| Eprino/Delta | 88.6 | 0.0 | 0.0 | 0.0 | 0.0 | |
| Negative | 110.0 | 91.9 | 87.4 | 85.1 | 57.4 | |

TABLE 9
Reduction compared to control (arithmetic means)

| | D 0 | D 7 | D 14 | D 31 | D 42 | D 56 |
|---|---|---|---|---|---|---|
| Eprino/Delta | NA | 100.0 | 100.0 | 100.0 | 100.0 | |
| Negative | NA | NA | NA | NA | NA | NA |

TABLE 10
Additional API Stress Data

| Batch | | Mectin | Pyrethroid | | | Viscosity |
|---|---|---|---|---|---|---|
| No. | Formulation | 5 Days 70° C. | 5 Days 70° C. | Density | pH | (cps) |
| 45 | Solution | Doramectin - | Deltamethrin - | 0.927 | n/a | 36 |
| | | 96.1% | 99.7% | | | |
| 48 | Suspension | Doramectin - | Deltamethrin - | 1.044 | 5.68 | 135 |
| | | 99.3% | 98.9% | | | |
| 46 | Solution | Moxidectin - | Cypermethrin - | 0.926 | n/a | 34 |
| | | 102.5% | 100.5% | | | |
| 49 | Suspension | Moxidectin - | Cypermethrin - | 1.046 | 5.83 | 136 |
| | | 99.4% | 98.3% | | | |
| 55 | Solution | Ivermectin - | Flumethrin - | 0.926 | n/a | 35 |
| | | 100.4% | 97.7% | | | |
| 56 | Suspension | Ivermectin - | Flumethrin - | 1.049 | 5.91 | 128 |
| | | 100.4% | 99.4% | | | |

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A pharmaceutically or veterinarily acceptable formulation adapted to be topically applied to an animal, which formulation comprises;
a. an effective amount of a pyrethroid;
b. an effective amount of at least one macrocyclic lactone;
c. a pharmaceutically acceptable system of solvents and/or suspending agents and excipients wherein the system provides for acceptable storage stability for both the pyrethroid and the macrocyclic lactone.

2. The formulation of claim 1 wherein the macrocyclic lactone is an avermectin, abamectin, doramectin, eprinomectin, ivermectin, moxidectin, milbemycin, milbemectin, latidectin, or combinations thereof, and the pyrethroid is cyfluthrin, cypermethrin, deltamethrin, flumethrin, or combinations thereof.

3. The formulation of claim 2 wherein the macrocyclic lactone is dissolved in an organic solvent and the pyrethroid is suspended in an aqueous phase.

4. The formulation of claim 3 wherein the organic solvent is propylene glycol.

5. The formulation of claim 4 wherein the propylene glycol is present in an amount between 45 and 65 percent (w/v).

6. The formulation of claim 5 wherein the propylene glycol is present in an amount between about 50 and 60, or between about 53 and 57, or is about 55 percent (w/v).

7. The formulation of any of claims 4-6 further comprising a preservative, a buffer, a surfactant, an anti-foaming agent, and an anti-oxidant.

8. The formulation of claim 7 wherein the preservative is BHT, methyl paraben sodium, propyl paraben sodium, or combinations thereof, the buffer is citric acid, and the surfactant is POLYSORBATE 80.

9. The formulation of claim 8 further comprising a defoamer, an anti-caking agent, a suspending agent, an pacifier, and a colouring agent.

10. The formulation of claim 9 wherein the anti-caking agent is AEROSIL 200, the suspending agent is Xanthum gum, the opacifier is titanium dioxide, and the colouring agent is FD &C Yellow number 5 alum lake.

11. The formulation of claim 1 wherein the solvent is a neutral oil and the macrocyclic lactone is present in an amount between 0.2 and 0.8 (w/v) and the pyrethroid is present in an amount between 0.4 and 1.0 (w/v), and both actives are dissolved in the oil.

12. The formulation of claim 11 wherein the oil is propylene glycol dicaprylate/dicaprate (MIGLYOL 840), the macrocyclic lactone is eprinomectin, and the pyrethroid is deltametrin.

13. A process for making the formulation of any one of claim 1, 11 or 12 comprising the steps of:
a. loading 95% MIGLYOL 840 & eprinomectin;
b. adding deltamethrin with mixing until the solution is clear; and
c. bringing to final volume with MIGLYOL 840;
d. thereby making the formulation.

14. A process for making the formulation of any one of claim 1-6, or 8-10 comprising the steps of:
a. adding the solvent to a suitable vessel;
b. adding the macrocyclic lactone and an antioxidant to the vessel and mixing until a clear solution is obtained;
c. preparing a preservative solution comprising the steps of:
i. Dispensing Purified water (30% of batch volume) in a vessel; and
ii. Adding Methyl Paraben Sodium and Propyl Paraben Sodium to step (a) with mixing, mix till clear solution achieved;
d. adding the preservative solution of step c to the solution of step b and mixing with homogenization;
e. adding citric acid anhydrous and POLYSORBATE 80 to the mixture of step d and mixing until a clear solution is achieved;
f. adding defoamer to mixture of step e;
g. adding deltamethrin to the mixture of step f to produce a first dispersion;
h. adding AEROSIL 200 to the dispersion of step g to produce a second dispersion;
i. adding in a second vessel propylene glycol and xanthum gum to form a third dispersion;
j. adding the third dispersion of step i to the second dispersion of step h and mixing to form a forth dispersion;
k. optionally adding opacifiers and/or colourants; and bringing to final volume with water, mixing with homogenization thereby making the formulation.

15. A formulation according to any one of claim 1-6, or 8-10, wherein milk withdrawal time is less than 2 weeks.

16. The formulation of claim 15 wherein the milk withdrawal time is less than 10 days.

17. The formulation of claim 15 wherein the milk withdrawal time is less than 9 days.

18. The formulation of claim 15 wherein the milk withdrawal time is less than 8 days.
